(19) United States
(12) Patent Application Publication
(10) Pub. No.: US 2023/0047464 A1
(43) Pub. Date: Feb. 16, 2023

(54) ELECTROLYTIC CHARGED CRYSTALLINE NANOMETRIC IONS

(71) Applicant: Shawn Hill, Henderson, NV (US)

(72) Inventor: Shawn Hill, Henderson, NV (US)

(21) Appl. No.: 17/883,172

(22) Filed: Aug. 8, 2022

Related U.S. Application Data
(60) Provisional application No. 63/231,440, filed on Aug. 10, 2021.

(51) Int. Cl. A61K 33/38; A61K 33/00; A61K 41/00

(52) U.S. Cl. CPC A61K 33/38; A61K 33/00; A61K 41/00

(57) ABSTRACT

An antimicrobial composition that includes nanoparticles of silver and titanium. The composition can further include boron, silica, selenium, vitamin, minerals, and the like or combinations thereof.

### CROSS-REFERENCE TO RELATED APPLICATIONS

This application claims priority from a U.S. Provisional Patent Appl. No. 63/231,440 filed on Aug. 10, 2021, which is incorporated herein by reference in its entirety.

### FIELD OF INVENTION

The present invention relates to an antimicrobial composition, and more particularly, the present invention relates to an antimicrobial composition that has a combination of silver and titanium nanoparticles.

### BACKGROUND

Silver is traditionally being used in medicine for its antimicrobial properties for hundreds of years. OTC medical products containing silver nitrate are quite popular for the treatment of burns and ulcers. Colloidal silver forms are widely used in alternative medicines for the treatment of bacterial, fungal, and viral infections.

Despite several clinical uses reported for colloidal silver, the use of colloidal silver is limited to wound dressings, burns, ulcerations, and the like. However, the increasing antibiotic resistance has renewed the interest in silver for its antimicrobial properties. Silver nanoparticles are known in the art as having few advantages over colloidal silver. Nano formulations of silver are widely used for antimicrobial and antiviral textile treatments. Many healthcare products such as bandages, ointments, and cleaning solutions contain nano silver. However, the nano silver particles also have the limited mode of action against infections.

Thus, considering the potential clinical applications of silver, a need is appreciated for an improved composition of silver.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a composition having a combination of silver and titanium nanoparticles, which has a broad antimicrobial application.

It is another object of the present invention that the bioavailability of the composition can be improved.

In one aspect, disclosed is a piezoelectric mechanical process to create “Electrolytic Charged Crystalline Nanometric Ions.”

In one aspect, disclosed is a method of producing an antimicrobial composition, the antimicrobial composition comprises silver nanoparticles and titanium nanoparticles, the method comprises the steps of subjecting one or more silver rods to an electrolysis process using a predetermined voltage and a predetermined frequency to obtain the silver nanoparticles; subjecting one or more titanium rods to the electrolysis process using the predetermined voltage and the predetermined frequency to obtain the titanium nanoparticles; and combining the silver nanoparticles and the titanium nanoparticles to obtain a solution. The predetermined voltage is 27 V DC voltage, and the predetermined frequency is 5284 Hz. The method further comprises adding boron and silica to the solution.

In one aspect, disclosed is an antimicrobial composition comprises silver nanoparticles and titanium nanoparticles, the antimicrobial composition is prepared by a method comprising subjecting one or more silver rods to an electrolysis process using a predetermined voltage and a predetermined frequency to obtain the silver nanoparticles; subjecting one or more titanium rods to the electrolysis process using the predetermined voltage and the predetermined frequency to obtain the titanium nanoparticles; and combining the silver nanoparticles and the titanium nanoparticles to obtain a solution. The predetermined voltage is 27 V DC voltage, and the predetermined frequency is 5284 Hz. The method further comprises adding boron and silica to the solution.

An antimicrobial composition comprising silver nanoparticles; and titanium nanoparticles.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a geometric arrangement of crystalline solids formed by the application of 5284 Hz, according to an exemplary embodiment of the present invention.

FIG. 2 shows a geometric arrangement of crystalline solids formed by the application of 345 Hz, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described, wherein like reference numerals are used to refer to like elements throughout. In the following description, for purposes of explanation, specific details may be set forth to provide a thorough understanding of the subject innovation. It may be evident, however, that the claimed subject matter may be practiced without these specific details. In other instances, well-known structures and apparatus are shown in block diagram form to facilitate describing the subject innovation.

Disclosed is a composition that includes a combination of silver and titanium nanoparticles. The composition can further include silica, boron, and selenium in any suitable form.

Titanium and its oxides, in an aqueous environment, have a low ion-formation tendency and low reactivity with macromolecules such as viruses. Titanium is biocompatible because of its low electrical conductivity and corrosion resistance. Titanium is a non-ferromagnetic transition metal that contains unpaired electrons. Titanium forms an inert oxide layer that spontaneously forms when its surface is exposed to oxidizing media such as geodesic dome coronaviruses.

The silica tetrahedron is the basis for all the silicate structures. SiO₄ tetrahedron has a neutral charge. Characterized by independent SiO₄ tetrahedra, which are not linked together directly. They are bonded together by ionic bonds to interstitial cations. The structures of the nesosilicates are, therefore, very dependent on the size and charge of the interstitial cations because the tetrahedral “do not share oxygen,” the Si:O ratio is 1:4.

Boron has good antiseptic, antifungal, and antiviral properties as an effective and inexpensive competitor to expensive synthetic “amorphous” pharmaceutical drugs. It has a vital role in converting vitamin D to its active form. Boron also helps to rid the body of fluoride accumulation and toxic heavy metals.

Disclosed is a tetrahedron crystalline transitional matrix that includes a combination of titanium and silver nanoparticles. The composition can further include boron, silicon, selenium, or the like elements or a combination of two or more of such elements. The composition can further include minerals and vitamins. The amount of such elements, minerals, and vitamins can be within the prescribed limits. For example, within the RDA (Recommended Daily Allowance) limits. Each respective element and/or ingredient can be added to any water-soluble solution based upon its own “biochemical uniqueness” as such that will produce a different chemical bond to carry out a non-spontaneous chemical reaction.

In one implementation, disclosed is a tetrahedron crystalline transitional matrix formed by the nanoparticles of titanium and silver. The nanoparticles of titanium and silver can be produced by electrolysis and thus are electrolytically charged. This unique tetrahedron crystalline transitional matrix provides a combination of the highest specific surface area and microporosity. The combined use of: Ag₄₇, Ti₂₂, B₅, & Si₁₄, increases the bonding efficiency obtained, and achieves higher bonding efficacy than that obtained using colloidal silver (Ag₄₇) alone. The use of combined silver and titanium nanoparticles together can act as nanocatalysts that promote a chemical reaction that increases the rate of oxygen absorption without these inert elements themselves undergoing any permanent chemical change.

The advantages of the use combination of electrolytic charged crystalline nanometric ions allow bonding to occur whereby these enhanced ions can be used for “in vivo” localized treatments where “crystalline nanometric ions” can freely circulate throughout the bloodstream and be properly phagocytosed (bound) by cells without losing electrons due to “in vivo supersaturation”.

Liquid Boron i.e., ionic boron from ionized sodium borate (Na₂H₂₀B₄O₁₇) in water, the concentration of which is approximately 2.5% by weight. Liquid silica i.e., silicon dioxide solution of silicic acid (Si(OH)₄) in water—the concentration of which is approximately 2.5% by weight (measured as silica, SiO₂). Liquid Selenium, (i.e., Ionic Selenium from ionized Selenite), Na₂SeO₃ in water—the concentration of which is approximately 2.5% by weight.

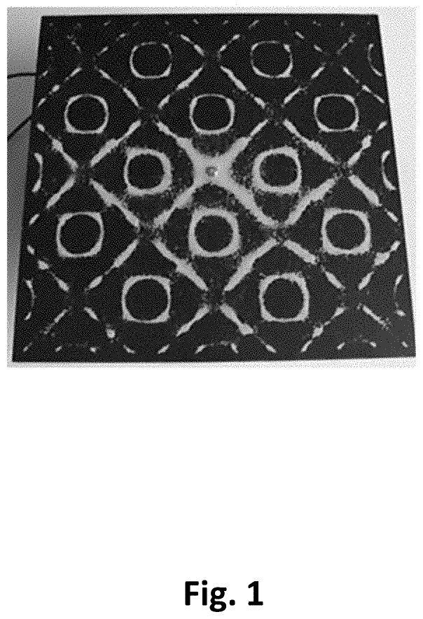

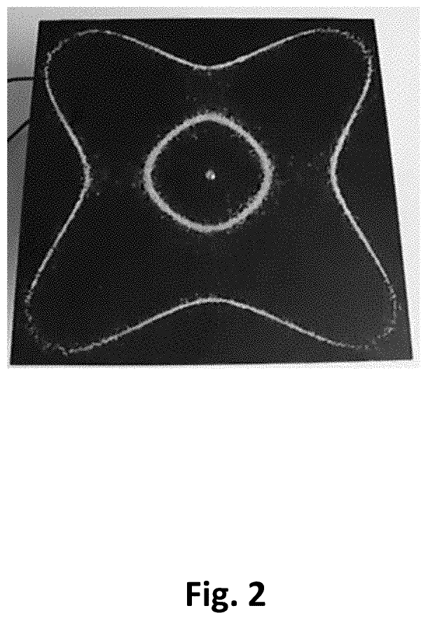

The disclosed composition can be produced by electrolysis, wherein the silver and titanium nanoparticles can be electrolytically charged. Pure silver and titanium rods can be used to generate silver and titanium nanoparticles. The low-frequency signal tuned to 110 to 5284 Hz can be applied to the rods via a series circuit during the electrolysis. The effect of applying a frequency vibrational rate of 5284 Hz creates the geometric arrangement of crystalline solids as shown in FIG. 1 while a frequency vibrational rate of 110 Hz can create the geometric arrangement of crystalline solids as shown in FIG. 2.

### Example

LifeForce Colloidal Silver Generators, 3.66 mm Silver Rods, 2 mm Titanium Rods, 24 Oz. Distilled Water, INTLLAB Magnetic Stirrer, and DDS Low Frequency Signal Generator Sine Wave Output Tuned to: 5284 Hz were used in the experiment.

Distilled water is infused with electrically charged Silver and Titanium ions via the Electrolysis Process. Two Silver rods of 99.99% purity, 10-gauge (3.6 mm in diameter) being 10 mm apart from each other in 709 ml distilled water as an electrolyte and connected to a 27-volt (DC) power supply. Two Titanium rods of grade 5, and of size 2-gauge (2.0 mm in diameter) being 10 mm apart from each other in 709 ml distilled water as an electrolyte and connected to a 27-volt (DC) power supply. A 27-volt DC electric current can be passed through the rods for electrolysis. A piezoelectric mechanical process can be used by applying a low frequency signal tuned to approximately 5284 Hz applied to the rods via a series circuit. The combined use matrix of crystalline nanometric ions that are frequency encoded utilizing a DDS Function Generator tuned at 5284 Hz provides a colloidal solution an “Electrolytic Charge.” Boron (B5) and Silicon (Si14) silica tetrahedrons were introduced to a distilled water colloidal solution via the ion exchange method and formed bonds with the metal ions to form a combined “matrix of crystalline nanometric ions.”

Both the electrolyte solutions were stirred at a constant rate of 100 rpm to homogenize the electrolyte and maintain a uniform current during the electrolysis process. The current through the distilled water as an electrolyte, two silver metallic rods and two titanium metallic rods were in separate glass containers, in turn, is linked with the ends of each having a separate power source.

The composition can be produced from the silver and titanium nanoparticles in any desired proportion. Liquid sodium, silicon, silicon, vitamin, minerals, alone or in combinations can be added.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method of producing an antimicrobial composition, the antimicrobial composition comprises silver nanoparticles and titanium nanoparticles, the method comprises:
subjecting one or more silver rods to an electrolysis process using a predetermined voltage and a predetermined frequency to obtain the silver nanoparticles;
subjecting one or more titanium rods to the electrolysis process using the predetermined voltage and the predetermined frequency to obtain the titanium nanoparticles; and
combining the silver nanoparticles and the titanium nanoparticles to obtain a solution.

2. The method according to claim 1, wherein the predetermined voltage is 27 V DC voltage.

3. The method according to claim 2, wherein the predetermined frequency is 5284 Hz.

4. The method according to claim 1, wherein the method further comprises:
adding boron and silica to the solution.

5. An antimicrobial composition comprises silver nanoparticles and titanium nanoparticles, the antimicrobial composition is prepared by a method comprising:
subjecting one or more silver rods to an electrolysis process using a predetermined voltage and a predetermined frequency to obtain the silver nanoparticles;
subjecting one or more titanium rods to the electrolysis process using the predetermined voltage and the predetermined frequency to obtain the titanium nanoparticles; and
combining the silver nanoparticles and the titanium nanoparticles to obtain a solution.

6. The antimicrobial composition according to claim 5, wherein the predetermined voltage is 27 V DC voltage.

7. The antimicrobial composition according to claim 6, wherein the predetermined frequency is 5284 Hz.

8. The antimicrobial composition according to claim 5, wherein the method further comprises:
adding boron and silica to the solution.

9. An antimicrobial composition comprising:
silver nanoparticles; and
titanium nanoparticles.
